(19) United States
(12) Patent Application Publication
(10) Pub. No.: US 2008/0039912 A1
(43) Pub. Date: Feb. 14, 2008

(54) THERMO BAG HEATING DEVICE

(76) Inventor: Lung-Wen Kuo, Taipei County 235 (TW)

Correspondence Address: SCHMEISER, OLSEN & WATTS, 22 CENTURY HILL DRIVE, SUITE 302, LATHAM, NY, 12110, US

(21) Appl. No.: 11/834,038

(22) Filed: Aug. 6, 2007

(30) Foreign Application Priority Data
Aug. 11, 2006 (TW) 095214237

(51) Int. Cl. A61B 18/04; H05B 1/00

(52) U.S. Cl. 607/108; 219/209

(57) ABSTRACT

A thermo bag heating device includes a housing internally defining a through receiving space; a heating basket being an open-topped hollow thermal conductive container disposed in the receiving space for receiving a thermo bag therein for heating; and a heating element wound around an outer surface of the heating basket for heating the same when an amount of electric current is supplied to the heating element. The heating element is serially connected at an end to a temperature controller and a fuse, and is connected via a control circuit board to a timer for setting duration of heat, a control switch for controlling on/off of the heating element, and a buzzer and a plurality of indicator lamps for emitting warning sound and light, respectively. A metal foil is wound around the heating basket to cover and locate the heating element in place while providing a heat diffusing effect.

### FIELD OF THE INVENTION

The present invention relates to a thermo bag heating device, and more particularly to a heating device for conveniently, safely, and efficiently heating a thermo bag.

### BACKGROUND OF THE INVENTION

In cold winter or at relatively cold areas, the proper use of a thermo bag may protect a user against losing body temperature and maintain the user's body in a comfortable state. The thermo bag is a useful healthcare device particularly to the aged, patients, and people with relatively poor resistance.

Among others, a relatively common and simple thermo bag includes a bag made of a soft and thermal conductive material and provided with at least one sealable liquid inlet. To use the thermo bag, hot water or ice water is poured into the bag via the liquid inlet, depending on actual need. With the thermal conductive material of the bag, the hot or ice water in the bag is able to change ambient temperature near the thermo bag and make a user feeling comfortable. However, the thermo bag with the above simple structure is not convenient for use because the user has to pour out the used water and refill the empty bag with required hot or ice water frequently.

Another type of thermo bag includes a sealed bag made of a soft and thermal conductive material. A type of thermal conducting liquid, such as salt water, is pre-filled in the sealed bag. A proper heating mechanism is also provided in the bag. To use the thermo bag, connect the heating mechanism to a power supply to directly heat the thermal conducting liquid in the bag to a certain temperature depending on different usages, such as hot compress, warm keeping, etc. However, since the heating mechanism and the thermal conducting liquid are simultaneously provided in the sealed bag, there are potential problems of a broken bag due to thermally expanded conducting liquid, a leaky heating mechanism, etc. to endanger the safe use of the thermo bag. Thus, it is not easy to ensure the safety in use of the above-structured thermo bag. Moreover, the thermal conducting liquid is corrosive to thereby adversely affect the normal usable life of the heating mechanism.

It is therefore tried by the inventor to develop an improved thermo bag heating device to eliminate the drawbacks existed in the application of the conventional thermo bags.

### SUMMARY OF THE INVENTION

A primary object of the present invention is to provide a thermo bag heating device that could be conveniently used to heat a thermo bag.

Another object of the present invention is to provide a thermo bag heating device that is effectively protected against hazards possibly caused by any failure thereof to ensure the safety in use thereof.

To achieve the above and other objects, the thermo bag heating device according to the present invention includes a housing internally defining a through receiving space; a heating basket being an open-topped hollow thermal conductive container disposed in the receiving space of the housing for receiving a thermo bag therein for heating; and a heating element wound around an outer surface of the heating basket for heating the heating basket when an amount of electric current is supplied to the heating element. The heating element is serially connected at an end to a temperature controller and a fuse for cutting off the electric current at different temperature levels. And, a metal foil is wound around the heating basket to cover and locate the heating element in place while providing a heat diffusing effect.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an exploded perspective view of a thermo bag heating device according to a first embodiment of the present invention;

FIG. 2 is an assembled view of FIG. 1;

FIG. 3 is a sectional view taken on line A-A of FIG. 2;

FIG. 4 is an exploded perspective view of a thermo bag heating device according to a second embodiment of the present invention; and

FIG. 5 is an assembled vertical sectional view of the thermo bag heating device of FIG. 4.

### DETAILED DESCRIPTION OF THE PREFERRED EMBODIMENTS

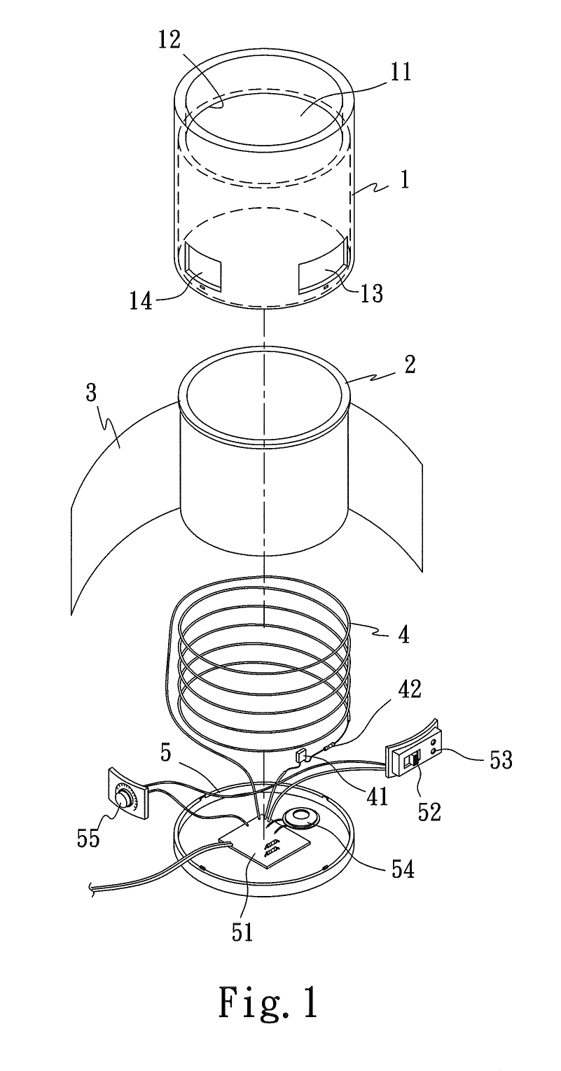

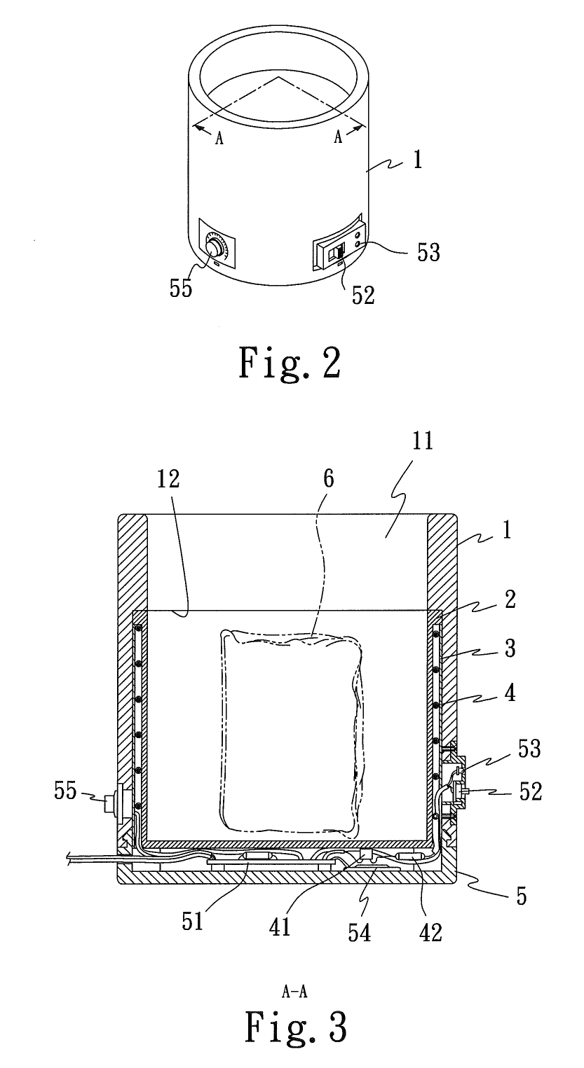

Please refer to FIGS. 1 and 2 that are exploded and assembled perspective views, respectively, of a thermo bag heating device according to a first embodiment of the present invention, and to FIG. 3 that is a sectional view taken on line A-A of FIG. 2. As shown, the thermo bag heating device according to the first embodiment of the present invention includes a housing 1, a heating basket 2, a metal foil 3, and a heating element 4.

The housing 1 is an electrically and thermally insulating container internally defining a hollow receiving space 11 having communicable open upper and lower ends. An upper portion of the receiving space 11 has a reduced inner diameter, so that an annular inner shoulder portion 12 is formed at a joint of the upper portion and a middle-to-lower portion of the receiving space 11. The housing 1 is provided near the lower end at predetermined positions with a first and a second opening 13, 14.

The heating basket 2 is a hollow and open-topped thermal conductive container for fitly positioning in the middle-to-lower portion of the receiving space 11, such that an upper end of the heating basket 2 is upward abutted on the inner shoulder portion 12 in the housing 1.

The heating element 4 may be a heating wire or any other element that produces heat when being supplied with an amount of electric current. The heating element 4 is wound around or attached to an outer surface of the heating basket 2, such that the heating basket 2 is directly heated when the heating element 4 is supplied with electric current. A temperature controller 41, either a turning knob type or a pushbutton type, is connected in series to an end of the heating element 4; and a fuse 42 is serially connected between the heating element 4 and the temperature controller 41.

The metal foil 3 is wound around the heating basket 2 to cover the heating element 4, so as to locate the heating element 4 in place while providing the function of conducting and diffusing heat. In a preferred embodiment of the present invention, the metal foil 3 is an aluminum foil.

A base 5 is closed onto the open lower end of the housing 1, so that the heating basket 2 is stably held between the base 5 and the inner shoulder portion 12. On an inner side of the base 5, there is provided a control circuit board 51, via which a control switch 52, a plurality of indicator lamps 53, a buzzer 54, and a timer 55 are connected to the heating element 4. Wherein, the control switch 52 and the timer 55 are fitted in and accessible via the first and the second opening 13, 14 on the housing 1, respectively.

To user the heating device of the present invention to heat a thermo bag 6, first dispose the thermo bag 6 in the heating basket 2, and turn on the control switch 52 to start heating the heating element 4. The timer 55 may be set to control duration of heat for the heating element 4. The indicator lamps 53 and the buzzer 54 may produce light and sound, respectively, at different heating conditions to warn a user. For example, when the temperature controller 41 detects that the ambient temperature near the heating element 4 has reached a preset cut-off temperature, the current supplied to the heating element 4 is automatically cut off, and a suitable one of the indicator lamps 53 and the buzzer 54 work simultaneously or separately to produce light and sound, respectively, to warn the user. In the event the temperature controller 41 has problem and fails to timely cut off the current to the heating element 4, and the heating element 4 is kept heating to result in continuously rising ambient temperature, the fuse 42 would permanently cut off the power supply at another relatively higher temperature to ensure the safety in use of the thermo bag heating device of the present invention.

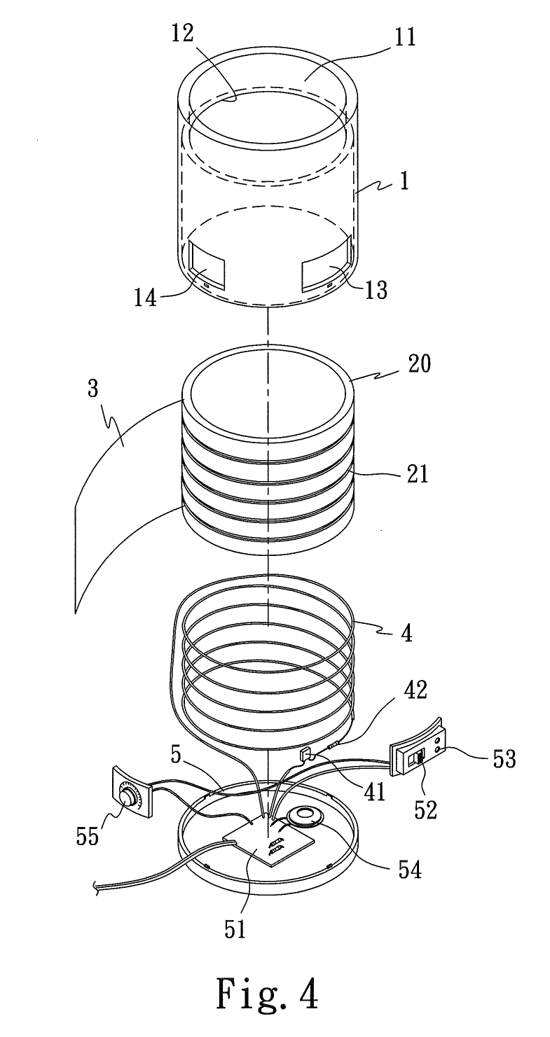

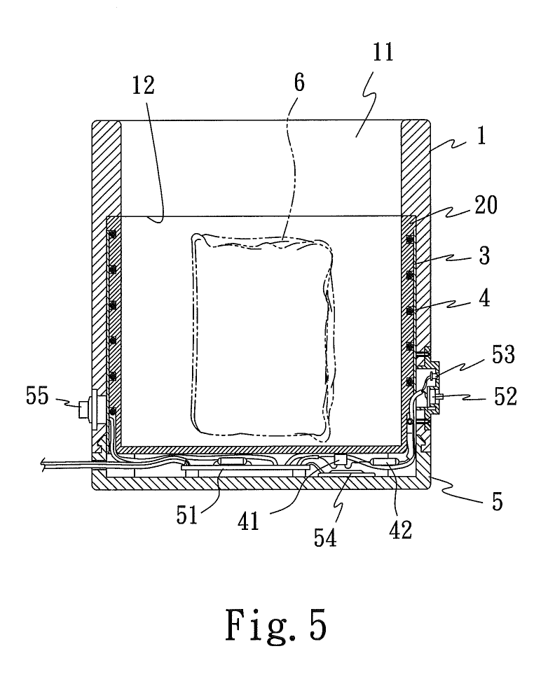

FIGS. 4 and 5 are exploded perspective view and assembled vertical sectional view, respectively, of a thermo bag heating device according to a second embodiment of the present invention. As shown, the second embodiment is generally structurally similar to the first embodiment, except for a heating basket 20 that is provided on and around an outer surface with a plurality of continuous wire grooves 21, in and along which the heating element 4 in the form of a heating wire is wound around the heating basket 20. This design ensures that the heating element 4 is well located around the heating basket 20 without the risk of displacement.

What is claimed is:

1. A thermo bag heating device, comprising:
a housing being an electrically and thermally insulating container internally defining a through receiving space;
a heating basket being an open-topped hollow and thermal conductive container for fitly positioning in a middle-to-lower portion of the receiving space of the housing;
a heating element being attached to an outer surface of the heating basket, such that when an amount of electric current is supplied to the heating element for the same to produce heat, the heating basket is directly heated by the heating element; and a temperature controller being connected in series to an end of the heating element and a fuse being serially connected between the heating element and the temperature controller to separately cut off the electric current at two different temperature levels; and
a metal foil being wound around the heating basket to cover the heating element.

2. The thermo bag heating device as claimed in claim 1, wherein the heating element is a heating wire, and the heating basket is provided on and around the outer surface with a plurality of wire grooves, in and along which the heating wire is wound around the heating basket.

3. The thermo bag heating device as claimed in claim 1, wherein the heating element is connected to a control circuit board, and connected via the control circuit board to a control switch and a plurality of indicator lamps; the control switch being used to control on/off of the heating element, and the indicator lamps to indicate different states of the heating element.

4. The thermo bag heating device as claimed in claim 3, wherein the heating element is further connected via the control circuit board to a timer for controlling duration of heat of the heating element.

5. The thermo bag heating device as claimed in claim 3, wherein the heating element is further connected via the control circuit board to a buzzer for emitting different warning sounds according to different heated states of the heating element.

6. The thermo bag heating device as claimed in claim 3, wherein the housing is internally provided at a joint of the middle-to-lower portion and an upper portion of the receiving space with an inner shoulder portion, and an open lower end of the housing is closed with abase; and the heating basket being held in the housing between the inner shoulder portion and the base.

7. The thermo bag heating device as claimed in claim 1, wherein the metal foil is aluminum foil.

8. The thermo bag heating device as claimed in claim 1, wherein the temperature controller is of a pushbutton type temperature controller.

9. The thermo bag heating device as claimed in claim 1, wherein the temperature controller is of a turning knob type temperature controller.
